(19) United States
(12) Patent Application Publication
(10) Pub. No.: US 2010/0266525 A1
(43) Pub. Date: Oct. 21, 2010

(54) METHOD FOR REMOVING MICROBES FROM SURFACES

(76) Inventors: Wilson Kurt WHITEKETTLE, Jamison, PA (US); Wenqing Peng, Shanghai (CN); Su Lu, Shanghai (CN); Juan Jiang, Shanghai (CN)

Correspondence Address: General Electric Company;GE Global Patent Operation, 2 Corporate Drive, Suite 648, Shelton, CT, 06484, US

(21) Appl. No.: 12/425,610

(22) Filed: Apr. 17, 2009

(51) Int. Cl. A01N 55/10; A01P 1/00

(52) U.S. Cl. 424/78.09

(57) ABSTRACT

A method has been found for the removal of microbial biofilm on surfaces in contact with systems, including but not limited to aqueous systems, which comprises adding to the aqueous system an effective amount of a carbosilane-based surfactant to substantially remove microbial biofilm, from surfaces in aquatic systems, while presenting minimal danger to non-target aquatic organisms at discharge due to their very low discharge concentrations.

### FIELD OF THE INVENTION

The field of the invention relates to methods for removing microbial biofilm from surfaces in contact with systems, including but not limited to aqueous systems. More particularly, the invention relates to the use of biodispersants in removal of microbial biofilm.

### BACKGROUND OF THE INVENTION

It is well known that bacteria attach to surfaces in any non-sterile aquatic environment. Industrial efforts to prevent colonization or to clean fouled surfaces amount to costly expenditures in many industries. Often such expenditures are made for cleaning programs that include the use of surfactants. Surfactants are regularly applied in water treatment programs as agents believed to play a role in the removal of organic masses from surfaces, in the enhancement of biocide efficacy or in the assistance in the water miscibility of various biocidal agents. Surfactants are also generally used in the agrichemical businesses, particularly to increase the effectiveness of herbicides. This is accomplished by using the surfactants to alter the surface area of the applied droplets, maximizing their interaction with leaf surfaces.

There are numerous examples of surfactants that inhibit the colonization of surfaces by inhibiting the overall growth of organisms in the growth target environment. Most surfactants, regardless of class, inhibit surface colonization when used in concentrations high enough to impede bacterial growth. In the water treatment industry, the most well known surfactants, which impart a measure of colonization resistance to submerged surfaces, include the cationic quaternary amine surfactants, which also function as biocides. Other surfactants, including anionic or non-ionic in chemical character, act to change the surface energy and prevent the microbes from attaching or growing at the water/surface interface. However, even the relatively mild nonionic or anionic surfactants can exhibit toxic effects upon microbes, such as bacteria, algae or fungi. The concentration of nonionic surfactants necessary to mediate toxicity is typically substantially higher than for cationic surfactants. Additionally, the more non-toxic surfactants often require higher levels of concentrations to achieve their purpose, thereby making them uneconomical, prone to forming high level of unwanted foam, and toxic to non-target aquatic organisms upon discharge to common receiving bodies of water.

One would expect nontoxic control of surface colonization to require the use of high concentration of surfactants, which is not possible in water treatment industries where thousands or millions of gallons of water would be treated. Accordingly, a need exists for a surfactant that can be used in water treatment industries, exhibiting low levels of toxicity when released into the environment, yet effective at low dosages to inhibit or remove biofilm in aqueous systems so there is an economical advantage.

### SUMMARY OF THE INVENTION

A method has been found for the removal or prevention of microbial biofilm on surfaces in contact with systems, such as but not limited to, aqueous systems, which comprises adding to the system an effective amount of Si-based surfactants, known as carbosilanes to substantially remove microbial biofilm, from surfaces in systems, while producing no stable foam and such that effluents discharged from the system present minimal danger to non-target aquatic organisms due to their very low discharge concentrations. The carbosilanes, also known as superspreaders, are ionic surfactants. This property gives the polysiloxanes the ability to readily infiltrate into the known water channels of exopolysaccharides (microbial biofilm) and disrupt the polysaccharide bonds that anchor the attached biomass to the submerged solid surfaces. The polyalkyleneoxide polysiloxanes show excellent compatibility with traditional oxidizing and non-oxidizing biocides as well. These surfactants can be used in conjunction with oxidizing biocides such as chlorine, bromine, halogenated hydantoins, chlorine dioxide, hydrogen peroxide, oxone, perborates, perchlorates, permanganates, as well as non-oxidizing biocides such as bronopol, isothiazolins, DBNPA, quaternary ammonium salts, methylene bis thiocyanate, dodecylguanidines, and others, to dislodge and disinfect surface-released biofilm masses. This type of combined treatment is actually preferred, as the carbosilane surfactant can greatly reduce the overall toxicity of the biocontrol program by reducing the amount of biocide needed for biofilm control. Additionally, due to the low dosage required, there are economical advantages as well.

The various features of novelty that characterize the invention are pointed out with particularity in the claims annexed to and forming a part of this disclosure. Changes to and substitutions of the various components of the invention can of course be made. The invention resides as well in sub-combinations and sub-systems of the elements described, and in methods of using them.

### BRIEF DESCRIPTION OF THE DRAWINGS

Refer now to the figures, which are meant to be exemplary and not limiting, and wherein like elements are numbered alike, and not all numbers are repeated in every figure for clarity of the illustration.

FIG. 1 is a chart depicting the results from biofilm removal efficacy in beaker test.

FIG. 2 is a chart depicting the results from biofilm removal at 50 ppm level for a 6 well plate test.

FIG. 3 is a chart depicting the results from biofilm removal at 50 ppm level for a 12 well plate test.

### DESCRIPTION OF THE INVENTION

Approximating language, as used herein throughout the specification and claims, may be applied to modify any quantitative representation that could permissibly vary without resulting in a change in the basic function to which it is related.

Accordingly, a value modified by a term or terms, such as “about”, is not limited to the precise value specified. In at least some instances, the approximating language may correspond to the precision of an instrument for measuring the value. Range limitations may be combined and/or interchanged, and such ranges are identified and include all the sub-ranges included herein unless context or language indicates otherwise. Other than in the operating examples or where otherwise indicated, all numbers or expressions referring to quantities of ingredients, reaction conditions and the like, used in the specification and the claims, are to be understood as modified in all instances by the term “about”.

As used herein, the terms “comprises,” “comprising,” “includes,” “including,” “has,” “having” or any other variation thereof, are intended to cover a non-exclusive inclusion. For example, a process, method, article or apparatus that comprises a list of elements is not necessarily limited to only those elements, but may include other elements not expressly listed or inherent to such process, method article or apparatus.

A method has been found for the removal or prevention of microbial biofilm on surfaces in contact with systems, such as but not limited to, aqueous systems, which comprises adding to the system an effective amount of Si-based surfactants, known as carbosilanes to substantially remove microbial biofilm, from surfaces in systems, while producing no stable foam and such that effluents discharged from the system present minimal danger to non-target aquatic organisms due to their very low discharge concentrations. In one embodiment of the present invention, the dispersant removes or reduces microbial slime from surfaces in contact with aqueous systems better than that caused by water alone. Microbial slime includes, but is not limited to, metabolizing cells plus exopolysaccharides that form an extra cellular mass in which the colony grows. Planktonic (free-floating) organisms may attach to surfaces of the aqueous system, exude various exopolysaccharides that may include natural surfactants, and gradually form a mat or film of like organisms. Such biofilm is to be avoided in aqueous systems as it may harbor pathogens, provide a niche for growth of anaerobic corrosion-causing microbes, reduce heat transfer across the surfaces or in other ways degrade the aqueous system. The dispersant performs this function without killing the microorganisms responsible for the adhesion. Therefore, this methodology has beneficial environmental effects, as it presents minimal danger to non-target aquatic organisms present in waste treatment systems or in other recipients of the discharge due to its very low discharge concentrations. Additionally, the dispersant according to an embodiment of the present invention does not cause excess amounts of foam that would be unacceptable in many aquatic systems. Waters treated in this manner are more acceptable for discharge to receiving streams having better aquatic toxicity profiles. Besides producing less foam, these carbosilane based dispersants do not provide significant nutrient value to microbes as many currently used organic carbon based surfactants do.

An embodiment of the present invention provides a method for removing microbial biofilm on surfaces in contact with systems, including but not limited to aqueous systems, comprising adding to the system an effective amount of dispersant comprised of carbosilanes. This can be accomplished by providing a composition comprised of a first hydrophobic moiety linked to a spacer that is linked to a second hydrophobic moiety. Hydrophobic moietys tend to be non-polar and thus prefer other neutral molecules and nonpolar solvents. Hydrophobic molecules in water often cluster together forming micelles. Water on hydrophobic surfaces will exhibit a high contact angle. Examples of hydrophobes include, but are not limited to alkanes, oils, fats and greasy substances in general.

In embodiments of the invention, the first hydrophobic moiety and the second hydrophobic moiety may be same hydrophobic moiety or they may be different hydrophobic moietys. For example, the first and second hydrophobic moietys may be comprised of organosilanes.

For such an embodiment, the first hydrophobic moiety and the second hydrophobic moiety may be comprised of organosilanes having formulae II, III, IV, or V

[R²₃Si]₂[R²₂Si]ₐ[R²SiH] (II)

[R²₃Si][R²₂Si]_{b}[R²₂SiH] (III)

[R³₃Si]₂[CH₂]ₐ[R³SiH] (IV)

[R³₃Si][CH₂]_{b}[R³₂SiH] (V)

wherein “a” is independently at each occurrence an integer from 0 to 50, “b” is independently at each occurrence an integer from 1 to 50, and R² and R³ are independently at each occurrence a C₁-C₃₀ aliphatic radical, a C₃-C₃₀ aromatic radical, or a C₃-C₃₀ cycloaliphatic radical.

In one embodiment of the composition defined above, the spacer is a hydrophilic moiety. As is widely known in the art a hydrophilic moiety or functional group is one that is typically charge-polarized and capable of hydrogen bonding, enabling it to dissolve more readily in water than in oil or other hydrophobic solvents. The hydrophilic moiety may be comprised of one or more of a cationic group, an anionic group, or a polar nonionic group, or combinations thereof. Further definition of hydrophobic groups would allow for them to include, but not be limited to an ammonium group, a phosphate group, a carboxylate group, a sulphate group, a peptide group, a carbohydrate group, a polyether, or combinations thereof.

An embodiment of the invention is also provided wherein the first hydrophobic moiety and the second hydrophobic moiety provide steric hindrance to a hydrolytic composition to protect the spacer from hydrolysis.

The dispersant comprises from about 20 to about 98 percent by weight of carboxysilane, with the remainder of the dispersant comprising water, which can be present in an amount of from about 2 to about 80% by weight. Additional components may include solvents, such as low molecular weight alcohols, for example, ethanol, methanol and butanol.

Carbosilane-based surfactants maintain performance over a broad range of pH systems, and are therefore advantageous for use in various aqueous systems. The carbosilane-based surfactants can be used in aqueous systems that have a pH of from about 3.5 to about 9.5.

The dispersant according to the present invention is preferably included in the aqueous system at a concentration of at least from about 2 parts per million (ppm) to about 400 ppm, with an alternative range of from about 20 to about 120 ppm, and a further embodiment of about 40 to about 60 ppm.

The systems that can be treated by the method and formulations disclosed herein are vast and varied, and may be any known systems involving chemical treatment for prevention and/or removal of microbial biofouling and macrofouling, particularly in aqueous based systems. Macrofouling as used herein is understood as involving larger organisms such as, but not limited to, shelled mollusks, hydrozoans, bryozoans, barnacles, sponges, and corals. As to industrial aqueous systems, the dispersant according to the present invention can be utilized in a variety of such systems, including but not limited to, commercial and industrial open recirculating cooling water towers, once-through and closed cooling water systems, cooling water conduits, heat exchangers, condensers, pasteurizers, air washers, heat exchange systems, air conditioning systems, humidifiers, dehumidifiers, hydrostatic cookers, safety and fire water protection storage systems, water scrubbers, disposal wells, influent water systems, including filtration and clarifiers.

In addition, the dispersants can be used in the treatment of wastewater, including, but not limited to wastewater treatment tanks, conduits, filtration beds, digesters, clarifiers, holding ponds, settling lagoons, canals, odor control systems, and ion exchange resin beds. With reference to membrane and filtration applications, the dispersants disclosed herein may be used in the treatment of membrane filtration, microfiltration, ultrafiltraton and nanofiltration membranes, reverses osmosis membranes and ultra pure water systems. In addition to the systems set forth above, other uses may include the use for example in food and beverage industries, for example in food and beverage disinfection systems.

The invention will now be described with respect to certain examples that are merely representative of the invention and should not be construed as limiting thereof.

### EXAMPLES

The invention is illustrated in the following non-limiting examples, which are provided for the purpose of representation, and are not to be construed as limiting the scope of the invention. All parts and percentages in the examples are by weight unless indicated otherwise.

In order to demonstrate efficacy of the present invention, a method was developed which allowed for the screening of dispersant ability to remove a bacterial biofilm. This method involved the colonization of commercially available 316 stainless steel coupons by bacteria, and their removal in the presence/absence of dispersants. The number of bacteria on a set of coupons was then determined by standard methods.

The bacterial species Pseudomonas fluorescens was chosen for these studies as this species is one that is common on submerged surfaces, and therefore would be one that could be expected to be found in process water streams.

The biofilm attached to the 316 stainless steel was formed by starting a 5 ml culture of Pseudomonas fluorescens in Nutrient Broth, it was incubated and shaken, overnight at 30° C. The next day, 1 ml of the culture was transferred into a 1.5 ml eppendorf tube. The culture was then placed in a centrifuge for 10 minutes at 10,000 g at 4° C. The liquid was decanted and the cell pellet resuspended in 0.85% sterile saline.

The transfer and centrifuge of the culture was repeated. Thereafter, Pseudomonas fluorescens cell pellet was resuspended in 1 ml of 0.85% sterile saline buffer and diluted with sterile saline buffer to OD₆₀₀ ˜0.050±0.02. A #4 Whattman filter paper was placed on top of all the Nutrient Broth plates needed, and 2 ml of prepared cell suspension was placed on top of each filter. Three 316 stainless steel coupons were placed on the filter paper of each Petri dish, and they were incubated at 30° C. for 24 hours. Biofilm was allowed to form on one side of the two sided coupons.

In order to show biodispersant treatment for biofilm coated coupons, on the third day, simulation cooling tower water was prepared and filtered to sterilization. A biodispersant stock solution (10,000 ppm) was prepared. Each beaker was filled with 700 ml cooling water and then an amount of cooling water was removed from each beaker equal to the amount of biocide/or dispersant that will be added to each particular beaker.

Appropriate amounts of biodispersant were added to each beaker at the concentration levels to be tested. The solutions were thoroughly mixed using the multi-stirrer. One beaker was maintained as a control and contained only 700 ml of simulation cooling water. Thereafter, three coupons with biofilm were aseptically placed on coupon holders, and then each coupon holder was placed into a slot in the coupon holder lid. Beakers were placed on a multi-stirrer and the stirring action was adjusted to mix the solution in the beaker gently for 24 hours.

35 ml sterile saline buffer were placed into 50 ml centrifuge tubes and one biofilm coupon was aseptically transferred into each centrifuge tube. Sonication was properly conduct in each tube to remove any remaining Pseudomonas fluorescens biofilm bacteria from each coupon and dispersed in a saline buffer.

Serial dilutions were performed using sterile saline buffer. Biofilm cell dilutions were inoculated on Petrifilm (3M Company). The Petrifilms are incubated at 30° C. for 48 hours, and the CFU (colony forming units) were read. Colony forming units (cfu)/cm² (Biofilm density) is determined by factoring the appropriate dilution and dividing the cell count obtained by 8.77 cm² (area of one side of a standard 316SS (stainless steel) corrosion coupon). The % of the biofilm removed was calculated by subtracting the above % calculation for each treatment from 100%. (biofilm controls minus treated).

(Optional calculation: % Reduction Achieved By Biodispersant=(Control Count-Treated Count)×100/Control Count)×100

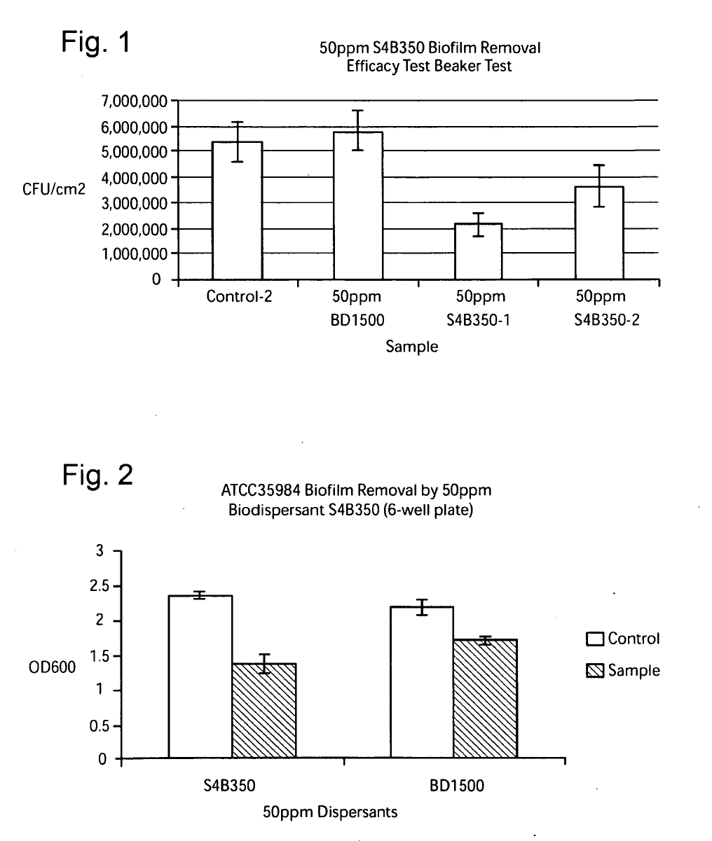

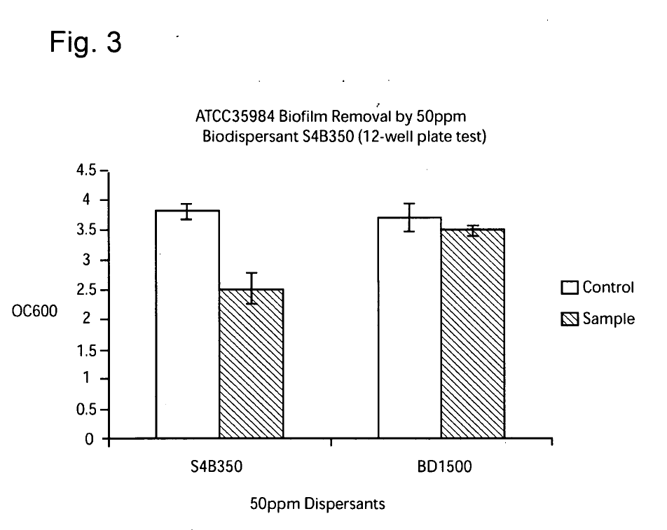

The results of the carbosilane-based dispersants on biofilm removal is shown in the charts below and the corresponding Figures. FIG. 1 relates to the results in Charts 1 and 2, FIG. 2 to Chart 3 and FIG. 3 to Chart 4. Results are shown for two different products, BD1500 (GE Betz, Trevose, Pa.) used as a benchmark, and S4B350, (Momentive Performance Materials, Wilton, Conn.) was the tested carbosilane chemical.

CHART I
Results of Carbonsilane on Biofilm Removal
Beaker-Test: 50 ppm BD1500 and S4B350 Biofilm Removal Efficacy Test

| | | | | cfu/cm2 | | SD/cfu/cm2 |
|---|---|---|---|---|---|---|
| | | | cfu/cm2 (average) | (average) | SD | (average) |
| control | Coupon1 | 1,370,000 | 5,467,503 | 5,347,777 | 785,098 | 14.7% |
| | Coupon2 | 1,520,000 | 6,066,135 | | | |
| | Coupon3 | 1,130,000 | 4,509,692 | | | |
| 50 ppm | Coupon1 | 1,360,000 | 5,427,594 | 5,813,379 | 738,402 | 12.7% |
| BD 1500 | Coupon2 | 1,340,000 | 5,347,777 | | | |
| | Coupon3 | 1,670,000 | 6,664,766 | | | |
| 50 ppm | Coupon1 | 450,000 | 1,795,895 | 2,155,074 | 460,251 | 21.4% |
| S4B350-1 | Coupon2 | 500,000 | 1,995,439 | | | |
| | Coupon3 | 670,000 | 2,673,888 | | | |
| 50 ppm | Coupon1 | 730,000 | 2,913,341 | 3,645,002 | 806,447 | 22.1% |
| S4B350-2 | Coupon2 | 1,130,000 | 4,509,692 | | | |
| | Coupon3 | 880,000 | 3,511,973 | | | |

CHART 2

| | cfu/cm2 | | |
|---|---|---|---|
| | (average) | SD | biofilm removal |
| control-2 | 5,350,000 | 780,000 | |
| 50 ppm | 5,810,000 | 740,000 | 0% |
| BD1500 | | | |
| 50 ppm | 2,160,000 | 460,000 | 60% |
| S4B350-1 | | | |
| 50 ppm | 3,650,000 | 810,000 | 32% |
| S4B350-2 | | | |

CHART 3
6-well plate biofilm removal test(ATCC35984 by
50 ppm biodispersant treatment)

| | Mean | SD | SD/Mean | |
|---|---|---|---|---|
| control | 2.408 | 2.3563333 | 0.045214305 | 1.9% |
| | 2.337 | | | |
| | 2.324 | | | |
| S4B350 | 1.517 | 1.374 | 0.128292634 | 9.3% |
| | 1.336 | | | |
| | 1.269 | | | |

CHART 4
12-well plate biofilm removal test(ATCC35984 by
50 ppm biodispersant treatment)

| | Mean | SD | SD/Mean | biofilm removal |
|---|---|---|---|---|
| control | 3.802666667 | 0.1296817 | 0.034102821 | |
| S4B350 | 2.518333333 | 0.2487053 | 0.098757901 | 33.80% |
| control | 3.711333333 | 0.2332216 | 0.062840392 | |
| BD1500 | 3.487 | 0.0818719 | 0.023479166 | 6.0% |

While the present invention has been described with references to preferred embodiments, various changes or substitutions may be made on these embodiments by those ordinarily skilled in the art pertinent to the present invention with out departing from the technical scope of the present invention. Therefore, the technical scope of the present invention encompasses not only those embodiments described above, but all that fall within the scope of the appended claims.

What is claimed is:

1. A method for removing microbial biofilm on surfaces in contact with a system, which comprises adding to the system a composition comprising a first hydrophobic moiety linked to a spacer which is linked to a second hydrophobic moiety.

2. The method of claim one wherein the spacer is a hydrophilic moiety.

3. The method of claim 2, wherein the hydrophilic moiety comprises a cationic group, an anionic group, a polar nonionic group, or combinations thereof.

4. The method of claim 3, wherein the hydrophilic moiety comprises one or more of an ammonium group, a phosphate group, a carboxylate group, a sulphate group, a peptide group, a carbohydrate group, or a polyether.

5. The method of claim 1 where the first hydrophobic moiety and the second hydrophobic moiety comprises organosilanes.

6. The method of claim 5, wherein the first hydrophobic moiety and the second hydrophobic moiety comprises organosilanes having formulae II, III, IV, or V
[R²₃Si]₂[R²₂Si]ₐ[R²SiH] (II)
[R²₃Si][R²₂Si]_{b}[R²₂SiH] (III)
[R³₃Si]₂[CH₂]ₐ[R³SiH] (IV)
[R³₃Si][CH₂]_{b}[R³₂SiH] (V)
wherein “a” is independently at each occurrence an integer from 0 to 50, “b” is independently at each occurrence an integer from 1 to 50, and R² and R³ are independently at each occurrence a C₁-C₃₀ aliphatic radical, a C₃-C₃₀ aromatic radical, or a C₃-C₃₀ cycloaliphatic radical.

7. The method of claim 1, wherein the first hydrophobic moiety and the second hydrophobic moiety provide steric hindrance to a hydrolytic composition to protect the spacer from hydrolysis.
